(19) United States
(12) Patent Application Publication
(10) Pub. No.: US 2025/0134773 A1
(43) Pub. Date: May 1, 2025

(54) METHOD AND SYSTEM FOR MANUFACTURING DOSING CAPSULES FOR CANNABIS-DERIVED RESIN

(71) Applicant: Capheads LLC, San Diego, CA (US)

(72) Inventor: Ori Lieberman, San Diego, CA (US)

(21) Appl. No.: 18/835,555

(22) PCT Filed: Feb. 3, 2023
(86) PCT No.: PCT/US2023/012321

§ 371 Date: Aug. 2, 2024

Related U.S. Application Data
(60) Provisional application No. 63/306,260, filed on Feb. 3, 2022.

(51) Int. Cl. A61J 3/07; A61K 9/48; A61K 31/00

(52) U.S. Cl. CPC A61J 3/077; A61K 9/4816; A61K 9/4833; A61K 31/658

(57) ABSTRACT

A method and system for manufacturing dosing capsules made of a cannabis-derived resin using a mold system, including using a dispensing plate having one or more reservoirs for holding a cannabis-derived resin powder, a first mold plate having one or more cavities corresponding to one or more reservoirs in the dispensing plate, and a second mold plate having one or more protrusions corresponding to one or more cavities in the first mold plate.

### CROSS-REFERENCE TO RELATED APPLICATION

This application is entitled to the benefit of and claims priority to co-pending U.S. Provisional Patent Application Ser. No. 63/306,260 filed Feb. 3, 2022 and entitled “Method And System For Manufacturing Dosing Capsules From Cannabis-Derived Resin.” The entire disclosure and contents of this prior filed application are hereby incorporated by reference herein in its entirety.

### TECHNICAL FIELD

The present disclosure relates to a method and system for manufacturing dosing capsules having a shell made from a crystallized cannabis-derived resin. The resin may have a known or predetermined potency of THCA, CBDA, or any other type of crystalline cannabinoid, or any other crystalline molecules found on cannabis such as terpenes and flavonoids. The system includes a dispensing plate with a reservoir and a mold.

### BACKGROUND

Crystallized cannabis-derived resins can be used to make a shell for a dosing capsule because they provide a structurally firm outer shell in which an inner material, such as another cannabis-derived resin, a pharmaceutical product, or any other ingestible material, may be encapsulated.

The physical properties of many cannabis-derived resins make it difficult to dispense a specific and uniform amount using traditional dispensing methods for use as a shell.

Further due to the properties, potencies, and consistencies of concentrates that can be used as inner material for a dosing capsule, it is not only hard to handle, but also difficult to determine the amount of dosage a consumer is taking. Traditionally, consumers of cannabis in products that vaporize various forms of cannabis concentrates (i.e., “dabbing”) have had to rely on imprecise estimates of Tetrahydrocannabinol (THC) content in the cannabis-related products, and no systems are in place that satisfactorily provide cannabis consumers a reasonable assurance of the THC content that they are ingesting.

### SUMMARY

Accordingly, there exists a need for a method of manufacturing dosing capsules from cannabis-derived crystalline resin powder that is easy to handle and may contain a known amount of inner material.

It is desired to provide a method and system that can quickly transfer melted resins into a mold before they start to cool and crystallize in the mold. It is also desired to provide a system in which excess resin can be collected after it is poured into a mold for reuse.

These and other needs are addressed by the present disclosure, which involves an improved method and system for manufacturing dosing capsules made of one or more cannabis-derived resins. The cannabis-derived resins may be made mostly of isolated cannabinoids, such as THCA (tetrahydrocannabinol acid extracted from trichomes), cannabidiolic acid (CBDA), cannabigerolic acid (CBGA), or any other cannabinoid resin, now known or later developed, whose physical and chemical makeup-allows it to crystallize or harden. The cannabinoids may be plant derived or bio-synthetized using yeast, bacteria, algae, or other microorganisms. In the present disclosure, cannabis derived resins also include hemp-derived resins as they also have a similar physical and chemical make-up and can be crystallized.

One aspect provides a method of manufacturing dosing capsules from a cannabis-derived resin using a dispensing plate having one or more reservoirs for holding a cannabis-derived resin, a first mold plate having one or more cavities corresponding to one or more reservoirs in the dispensing plate and a second mold plate having one or more protrusions corresponding to one or more cavities in the first mold plate. A first cannabis-derived resin is deposited into the reservoirs of the dispensing plate in resin powder form. The first cannabis-derived crystalline resin powder is heated within the reservoir such that it melts sufficiently to be poured into the first mold plate. The first mold plate is placed over the dispensing plate so that at least one reservoir of the dispensing plate registers with at least one cavity in the first mold plate. The dispensing plate and first mold plate are secured together and then rotated together such that the melted first cannabis-derived resin transfers from the reservoirs of the dispensing plate into the cavities of the first mold plate. The dispensing plate and first mold plate are then separated. A second mold plate containing at least one protrusion that corresponds with at least one cavity of the first mold plate is placed on top of the first mold plate such that the melted cannabis-derived resin is pressed by the protrusion to the wall of the reservoir. The resin is then cooled and forms a hardened, crystallized resin shell. The second mold plate is then removed from the first mold plate. The hardened shell may be filled with an inner material. After the shell is filled with an inner material, it may be covered by a melted second cannabis-derived resin to form a seal over the inner material. The second resin is then cooled and any excess amounts of the second cannabis-derived resin are removed, leaving a dosing capsule made of one or more cannabis derived resins.

The dosing capsules may have predetermined strengths, potencies, and volumes of cannabis and inner material, such that cannabis consumers can measure and consume precise doses of THCA, CBDA, CBGA or other cannabinoids, either by smoking, vaporizing, or by way of an edible product.

One aspect of the present disclosure is to provide a more accurate and easier to manipulate method for creating dosing capsules made from cannabis-derived resin.

The system allows for the volume of a dosing capsule to be more accurately specified and ensures uniformity among dosing capsules produced and ultimately consumed (by vaporizing, smoking, as well as consumer made edible products).

One aspect of the present disclosure includes a dispensing plate coated with polytetrafluoroethylene that is heated to a temperature that melts the cannabis-derived crystalline resin such that it can be transferred to a mold plate for shaping. The dispensing plate has a short-walled reservoir or cavity, i.e., a reservoir with a relatively short height, to allow heated cannabis-derived crystalline resin to spread evenly across the bottom surface of the reservoir to ensure quick and uniform heating of the resin. The dispensing plate may be coated with polytetrafluoroethylene or another material to ensure that once melted the cannabis-derived resin does not stick to the dispensing plate prior to and during transfer of the resin to a cavity in a mold.

Another aspect of the disclosure relies on a dispensing plate with a flask shaped reservoir that has a larger diameter toward the bottom and a smaller diameter toward the top to facilitate even heating and melting of the resin and to facilitate ease of transferring the resin into a cavity of the mold plate.

Another aspect of the disclosure relates to silicone mold plates to prevent the dosing capsule shell from cracking when it is hardened during the manufacturing process thus preventing waste and improving manufacturing efficiency.

Yet another aspect relates to a mold plate that has a cavity configuration that allows for easier trimming of excess material after the dosing capsule is sealed.

Yet another aspect relates to another mold plate that has a shallow recess configuration that allows for forming a seal of resin having a uniform height.

These and other aspects are provided by the method and system described herein.

### BRIEF DESCRIPTION OF THE DRAWINGS

FIG. 1 illustrates a perspective view of a dispensing plate having reservoirs used for manufacturing dosing capsules from cannabis-derived resin according to one embodiment of the present disclosure.

FIG. 2 illustrates a perspective view of a dispensing plate with a crystalline resin powder being deposited into a reservoir of the dispensing plate according to one embodiment of the present disclosure.

FIG. 3 illustrates a perspective view of a dispensing plate with cannabis-derived crystalline resin powder housed within reservoirs of the dispensing plate according to one embodiment of the present disclosure.

FIG. 4 illustrates a perspective view of a first mold plate above the dispensing plate of FIG. 3 according to one embodiment of the present disclosure.

FIG. 5 illustrates a side view of the dispensing plate and the first mold plate of FIG. 4 being connected and rotated together according to one embodiment of the present disclosure.

FIG. 6 illustrates a cross-sectional side view of the dispensing plate and the first mold plate connected after they have been rotated according to one embodiment of the present disclosure.

FIG. 7 illustrates a perspective view of the first mold plate after resin has been transferred into cavities in the first mold plate according to one embodiment of the present disclosure.

FIG. 8 illustrates a perspective view of a second mold plate above the first mold plate of FIG. 7 with resin in the cavities of the first mold plate according to one embodiment of the present disclosure.

FIG. 9 illustrates a cross-sectional side view of the first mold plate and second mold plate of FIG. 8, with the second mold plate in a position to mold the resin in the first mold plate according to one embodiment of the present disclosure.

FIG. 10 illustrates a perspective view of the second mold plate lifted above the first mold plate after the resin in the cavities of the first mold plate has been molded, cooled and hardened to form a shell according to one embodiment of the present disclosure.

FIG. 11 illustrates a perspective view of the first mold plate with a hardened cannabis-derived resin shell in the cavities of the first mold plate being filled with an inner material according to one embodiment of the present disclosure.

FIG. 12 illustrates a perspective view of the first mold plate with a hardened cannabis-derived resin shell and an inner material inside the shell, with a second cannabis-derived resin being added to seal the inner material inside the shell according to one embodiment of the present disclosure.

FIGS. 13 and 14 illustrate perspective views of the first mold plate and a laser being used to trim the material forming a seal on the shell according to one embodiment of the present disclosure.

FIG. 15 illustrates perspective views of two dosing capsules made according to one embodiment of the present disclosure.

FIG. 16 illustrates a cross-sectional side view of a cavity of the first mold plate with a peripheral reservoir surrounding the cavity according to one embodiment of the present disclosure.

FIG. 17 illustrates a perspective view of another example of the first mold plate with a number of cavities for receiving melted resin and peripheral reservoirs surrounding the cavities, with an enlarged view of a portion of one of the cavities according to another embodiment of the present disclosure.

FIG. 18 illustrates a perspective view of another example of the first mold plate with a number of cavities for receiving melted resin and a peripheral reservoir surrounding the cavities according to another embodiment of the present disclosure.

FIG. 19A illustrates a top plan view of a third mold plate according to one embodiment of the present disclosure.

FIG. 19B illustrates a cross-sectional side view of the third mold plate of FIG. 19A according to one embodiment of the present disclosure.

FIG. 19C illustrates a perspective view of the third mold plate of FIG. 19A according to one embodiment of the present disclosure.

### DETAILED DESCRIPTION

Hereinafter, embodiments of the present disclosure are described in detail with reference to the accompanying drawings. In the drawings, the same reference numerals or characters are used throughout to designate the same or equivalent elements of the disclosure. In addition, a detailed description of well-known elements, aspects, components, techniques, methods, systems, and the like associated with the present disclosure have been omitted in order not to unnecessarily obscure the gist of the present disclosure.

Terms such as first, second, A, B, (a), (b), and the like may be used herein to describe elements in embodiments of the present disclosure. These terms are only used to distinguish one element from another element. The intrinsic features, sequence or order, and the like of the corresponding elements are not limited by the use of these terms. Unless otherwise defined, all terms used herein, including technical or scientific terms, have the same meanings as those generally understood by those having ordinary skill in the field of art to which the present disclosure belongs. Such terms as those defined in a generally used dictionary are to be interpreted as having meanings consistent with the contextual meanings in the relevant field of art. Such terms are not to be interpreted as having ideal or excessively formal meanings unless clearly defined as having such in the present disclosure.

Specific structural and functional descriptions of the embodiments put forth in the present disclosure are illustrated only for the purpose of describing the embodiments according to the present disclosure. The embodiments according to the present disclosure may be embodied in various forms. The present disclosure should not be construed as being limited to only the specific form of the embodiments described herein. Since the embodiments according to the present disclosure may be variously changed and have various forms, specific embodiments are illustrated in the drawings and described in detail in the present specification or application. The embodiments and examples should be understood to include all changes, equivalents, and substitutes included in the spirit and scope of the present disclosure. Each embodiment herein is disclosed as including a specific set, number, grouping, arrangement, and/or the like of various aspects of the present disclosure. The present disclosure and claims are not intended to so limited, but instead may encompass embodiments that include different sets, numbers, groupings, and arrangements of the various aspects of the disclosure, which may be presently claimed or not presently claimed.

Various components, i.e., devices, units, elements, and the like of the present disclosure may be described herein as having a specific purpose or performing a function, step, set of instructions, process, or the like. Such components may be construed to be “configured to” achieve or meet the specific purpose or to perform the function, step, set of instructions, or process.

Traditional pills or capsules that contain a sought after (i.e., desired) ingestible product or material, such as a cannabis-derived resin, a pharmaceutical product or medication, or any other ingestible material that may be encapsulated such as a powder, oil or paste, use fillers or other materials to control the shape and/or form of the pill or capsule. However, the fillers or other materials used as the pill or capsule material adds to the total weight of the pill or capsule and are unnecessary, at least for certain products. The active ingredients of the desired product (cannabis product, medicine, etc.) may be listed, such the amount, in milligrams, of the desired product, but the total weight of the pill or capsule is not provided since the weight of the pill or capsule material is not relevant. The dosing capsules disclosed herein, however, are made up entirely of the desired product-cannabis-derived resin or concentrates. Thus, the outer shell is the desired consumption product as well as the inner material, and the filler or other material typically used to create the outer skins or shells of traditional pills or capsules can be eliminated. The result is a cleaner product containing only desired material to be consumed, where the total weight of the capsule is the total weight of the desired product (i.e., the desired dose). This eliminates the need for consumers to manually remove or dispense a desired quantity or weight of product prior to consuming, especially in situations where it may be necessary to heat the product prior to consumption, since heating traditional pill or capsule materials may cause unnecessary and unwanted side effects (e.g., smoking, burning, odor, residue, etc.).

The method and system for manufacturing dosing capsules made of one or more cannabis-derived resins disclosed and described herein solve or improve upon one or more of the above noted and/or other problems and disadvantages with prior known dosing capsules and systems and methods of manufacture. In one example, the disclosed dosing capsule, system, and method is easier to handle and can more accurately control the amount of inner material and overall weight and dimensions of the dosing capsule. In one example, the disclosed dosing capsule, system, and method can control the shape, size, and weight of the dosing capsule more precisely (using known densities) resulting in capsule dose and weight fluctuation within a much smaller range compared to the known dosing capsule and method of manufacture. In one example, the dosing capsule, system, and method can control the capsule size and shape based on densities to a degree that results in taking much less time to reach a desired dose and overall weight compared to the known dosing capsules and methods and systems of manufacturing the same. In one example, the disclosed dosing capsule, system, and method are configured to remove the produced capsule more easily from molds without damaging the capsule and to capture and reuse extra material during the manufacturing process. Thus, the manufacturing processes are easier, customizable, and more efficient. These and other objects, features, and advantages of the disclosure will become apparent to those having ordinary skill in the art upon reading this disclosure.

Turning now to the drawings, FIGS. 1-19 illustrate a method and system of creating a dosing capsule made of a cannabis-derived resin.

FIG. 1 shows a dispensing plate 100 for use in manufacturing a dosing capsule having a shell made of a cannabis-derived resin that can crystallize. The cannabis-derived resins disclosed herein may be made mostly of isolated cannabinoids, such as THCA (tetrahydrocannabinol acid extracted from trichomes), cannabidiolic acid (CBDA), cannabigerolic acid (CBGA), terpenes, or any other cannabinoid resin whose physical and chemical makeup-allows it to crystallize or harden.

The dispensing plate 100 has a number of reservoirs 102 for holding a resin, such as a cannabis-derived resin, to be molded into a capsule. As shown in FIG. 1, there are 25 reservoirs 102 configured in 5 rows and 5 columns. Other numbers of reservoirs and other configurations can be used.

Each reservoir 102 has a known volume. As shown, each reservoir 102 is defined by a generally cylindrical wall 104. As shown, the dispensing plate 100 contains uniform sized reservoirs 102 of a specific size, shape, diameter, and thickness. However, in other examples, the dispensing plate 100 could have several reservoirs 102 with different sizes, shapes and volumes. For example, reservoirs 102 in the dispensing plate 100 may be formed by a concave portion within the dispensing plate 100 or by a combination of a wall and a concave portion in the dispensing plate 100. Reservoirs 102 may also be flask-shaped with a larger diameter toward the bottom end of the reservoir 102 and a smaller diameter toward the top end of the reservoir 102. Reservoir shapes, such as flask-shaped reservoirs, may be designed to aid in the melting and transfer of resin, as discussed below.

The dispensing plate 100 may be made out of metal and coated with polytetrafluoroethylene. The dispensing plate 100 may also be made of materials such as silicone or glass or any other material that can withstand the heat needed to melt the cannabis-derived resin as described herein.

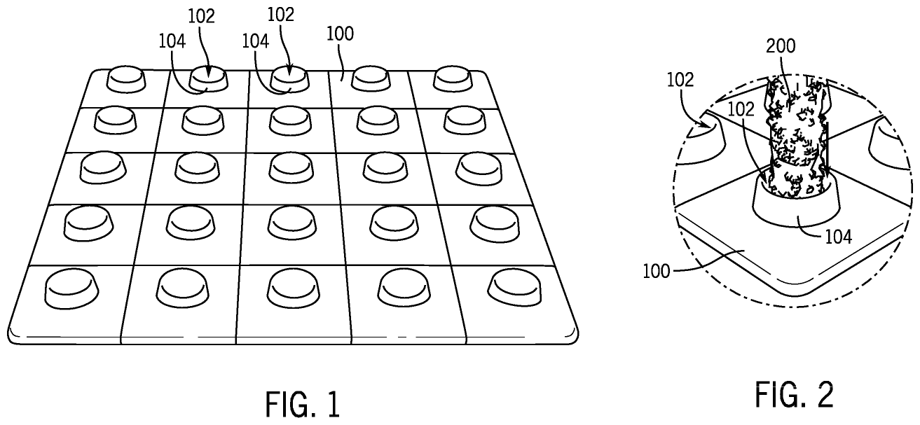

As shown in FIG. 2, a cannabis-derived crystalline resin 200 in powder form may be deposited into one of the reservoirs 102 of the dispensing plate 100. The powdered resin 200 can be deposited in one or more of the reservoirs 102 in a number of different ways. In one example, the dispensing plate 100 may be placed on an industrial sized scale to measure the precise weight of the cannabis-derived crystalline resin powder 200 that is deposited into given reservoirs 102 of the dispensing plate 100. Other means of depositing resin into the reservoirs using specific amounts, weights or volumes of the resin powder 200 may be used.

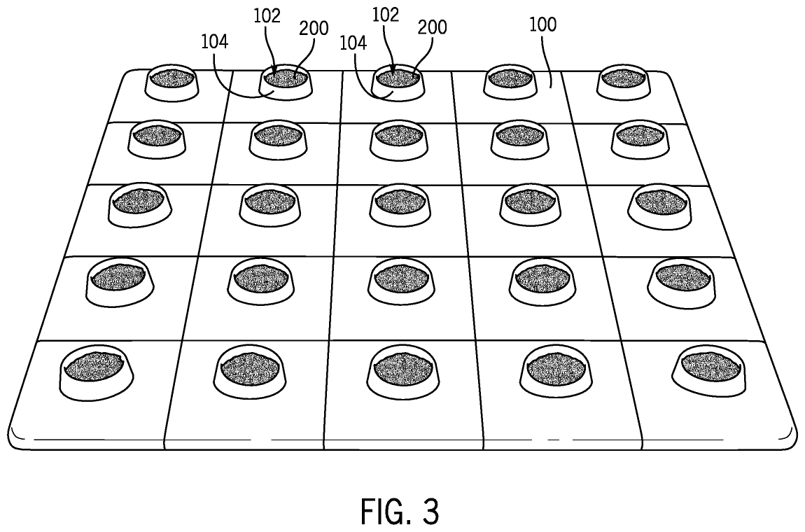

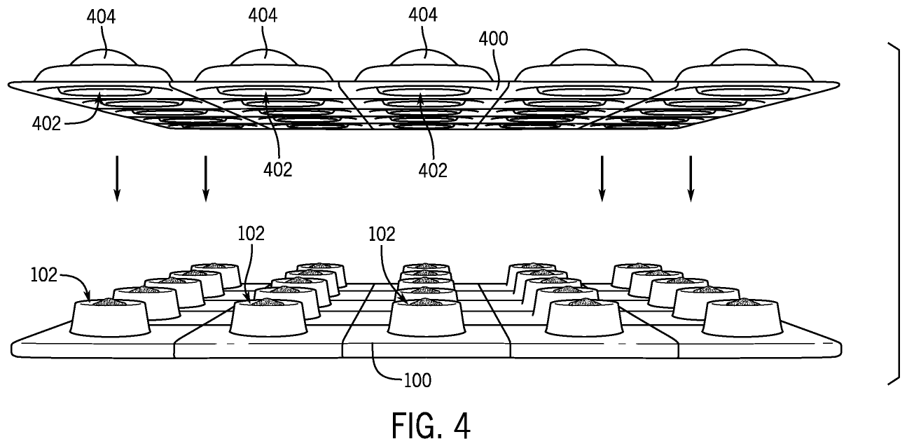

As shown in FIG. 3, a predetermined number of reservoirs 102 in the dispensing plate 100 have been filled with a predetermined amount of cannabis-derived crystalline resin powder 200. After the predetermined number of reservoirs 102 are filled with the desired amount of resin 200, the dispensing plate 100 may be heated to a temperature sufficient to melt the resin 200 to allow it to be poured from a reservoir 102 into a cavity in a mold (described below with reference to FIG. 4). Generally, for a cannabis-derived resin the temperature may be between 220-300 degrees Fahrenheit such that the cannabis-derived crystalline resin powder 200 inside each reservoir 102 begins to melt. However, melting points for different resins may vary. The size, shape, diameter and thickness of the reservoir 102 allows for quick and uniform melting of the cannabis-derived crystalline resin powder 200 within the cavities 102.

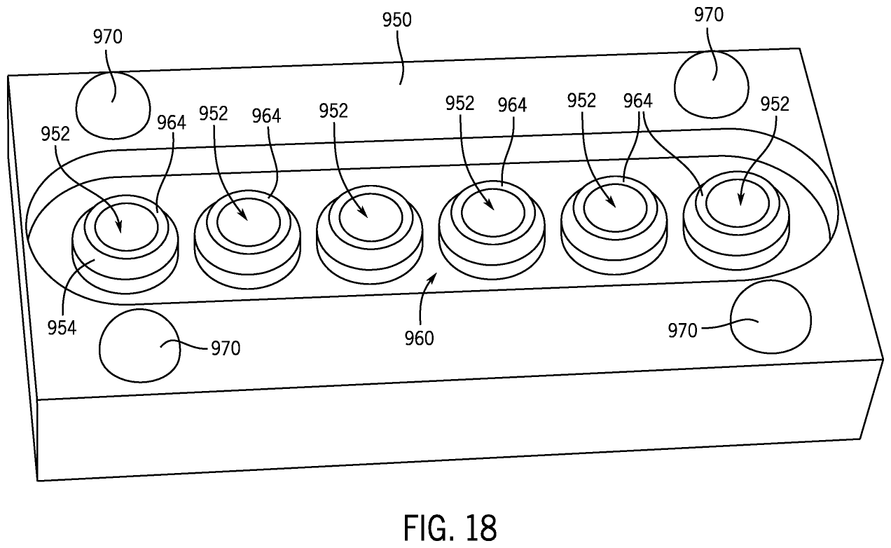

As shown in FIG. 4, a first mold plate 400 is provided. The first mold plate 400 may be made of a similar material as the dispensing plate 100 described above. As shown in FIG. 4, the first mold plate 400 has a number of cavities 402 defined by a rounded or spherical wall 404. However, in other examples, the first mold plate 400 could have several cavities 402 with different sizes, shapes and volumes. As shown in FIG. 4, the cavities 402 are placed in positions so that they can be placed over or in registration with the reservoirs 102 of the dispensing plate 100. As shown in FIG. 4, there are 25 cavities 402 in the first mold plate 400 and the cavities 402 are configured in 5 rows and 5 columns. In this way, the cavities 402 of the first mold plate 400 are positioned to correspond with all of the reservoirs 102 in the dispensing plate 100. Other numbers of cavities 402 and other configurations can be used. For example, as will be described below with reference to FIG. 18, the first mold plate 950 may have one row of six cavities 952.

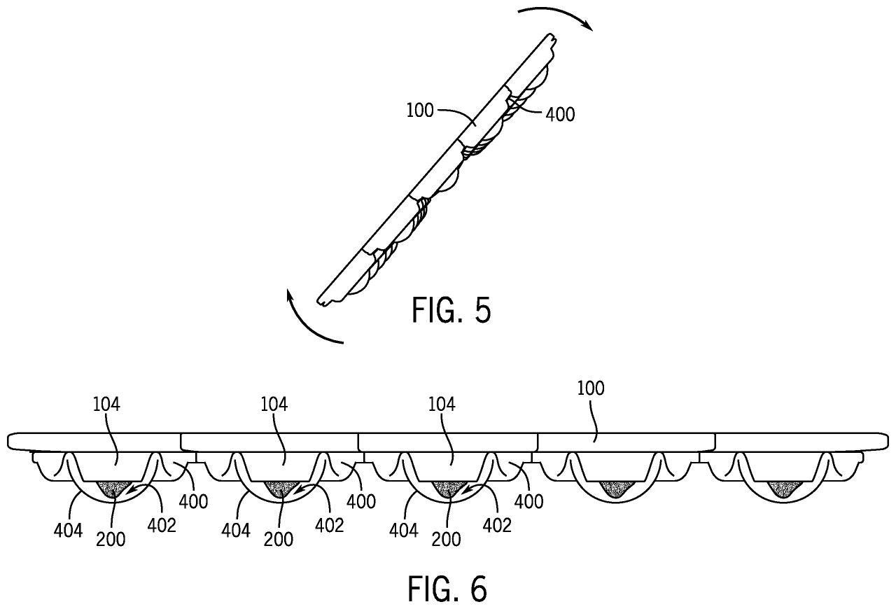

FIG. 5 shows the first mold plate 400 after it has been placed on top of the dispensing plate 100. As shown in FIG. 5, the dispensing plate 100 and the first mold plate 400 are held together and rotated along a generally horizontal plane, such that the heated, viscous, oily, or liquid resin 200 is transferred from the reservoirs 102 in the dispensing plate 100 to the cavities 402 in the first mold plate 400.

As shown in FIG. 6, after the dispensing plate 100 and the first mold plate 400 have been rotated and the dispensing plate 100 sits on top of the first mold plate 400, such that reservoirs 102 of the dispensing plate 100 correspond with the cavities 402 of the first mold plate 400, the melted cannabis-derived crystalline resin 200 flows or moves from the reservoirs 102 of the dispensing plate 100 into the cavities 402 of the first mold plate 400. In one example, and as shown in FIG. 6, the walls 104 of the dispensing plate 100 are configured such that the walls 104 do not touch the surface or walls 404 of the cavities 402 of the first mold plate 400.

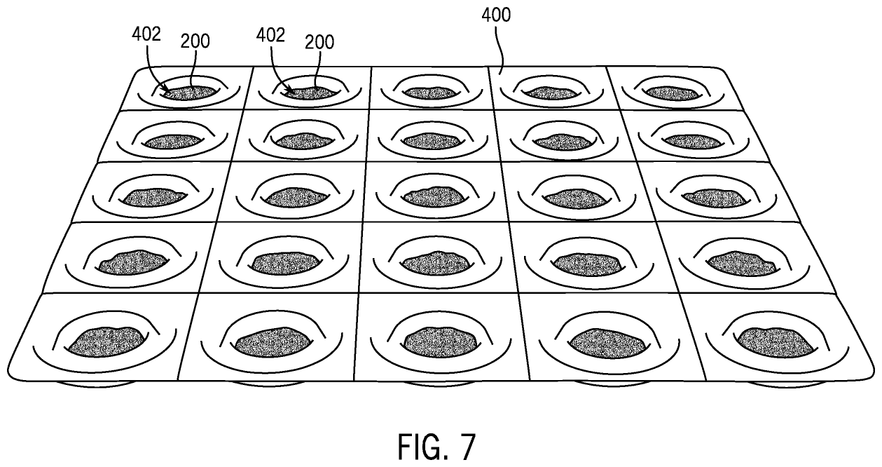

After the melted resin 200 has been transferred to the cavities 402 in the first mold plate 400, the dispensing plate 100 can then be removed or separated from the first mold plate 400. FIG. 7 shows the cavities 402 of the first mold plate 400 filled with the melted cannabis-derived resin 200 after the dispensing plate 100 is removed from the first mold plate 400. Since the melted cannabis-derived resin 200 may cool down and reharden quickly, the transfer of the resin 200 must be done quickly and while the resin 200 remains sufficiently heated to be able to flow to the cavities 402 of the first mold plate 400 from the dispensing plate 100.

In another example, rather than using the dispensing plate 100 described above, a predetermined amount of cannabis-derived crystalline resin powder 200 may be placed into one or more individual containers, such as a metal tube, and then heated to become melted cannabis-derived resin 200. In one example, the individual container may be heated using heating tape. Other heating methods may be used. Once the cannabis-derived resin 200 is melted, the melted resin 200 may be poured directly into the cavities 402 of the first mold plate 400. In one example, the heating and pouring steps may be done manually by hand. In another example, an automated dispensing system may be implemented to control the dispensing of the melted resin 200 into the cavities 402 of the first mold plate 400. The dispensing system may also be capable of heating the cannabis-derived crystalline resin powder 200 to become the melted resin 200.

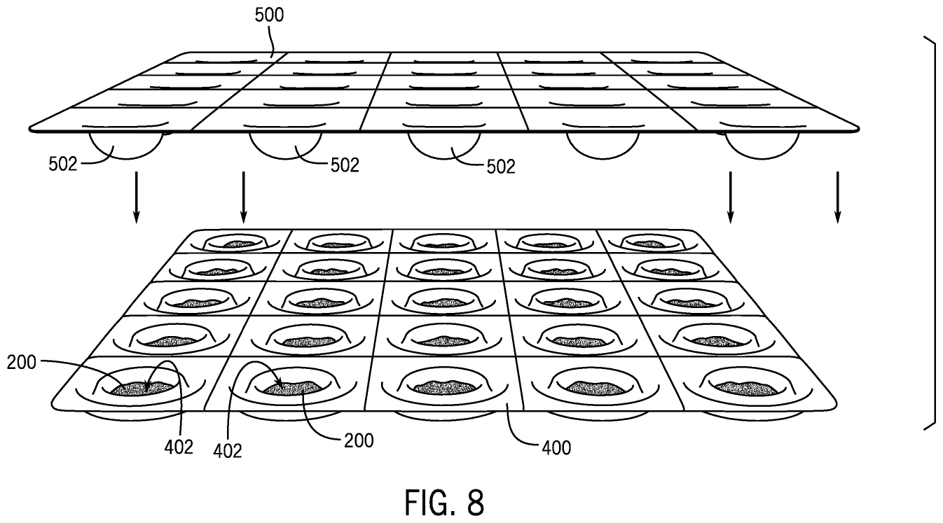

As shown in FIG. 8, a second mold plate 500 is provided. The second mold plate 500 may be made of the same or similar materials as the dispensing plate 100 and the first mold plate 400 described above. As shown in FIG. 8, the second mold plate 500 has a number of protrusions 502. As shown in FIG. 8, the protrusions 502 of the second mold plate 500 may be defined by a generally concave depressed wall. However, in other examples, the second mold plate 500 could have several protrusions 502 with different sizes, shapes and volumes.

As shown in FIG. 8, one or more of the protrusions 502 in second mold plate 500 correspond with the position and shape of one or more cavities 402 in the first mold plate 400. The second mold plate 500 of FIG. 8 has 25 protrusions 502 that are configured in 5 rows and 5 columns to correspond to the 5 rows and 5 columns of cavities 402 in the first mold plate 400. However, other numbers of protrusions 502 and other configurations can be used. For example, as will be described below with reference to FIG. 18, the second mold plate (not shown) may have one row of six protrusions to correspond to the one row of six cavities 952.

As shown in FIG. 8, the second mold plate 500 can be placed on top of the first mold plate 400 such that the protrusions 502 of the second mold plate 500 correspond with the cavities 402 of the first mold plate 400. The second mold plate 500 must be placed on top of the first mold plate 400 quickly and while the resin 200 remains sufficiently heated to be able to be molded and spread onto the walls 404, as described below.

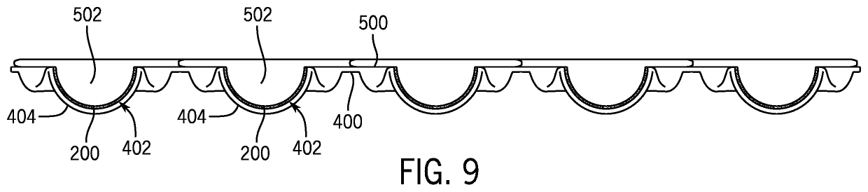

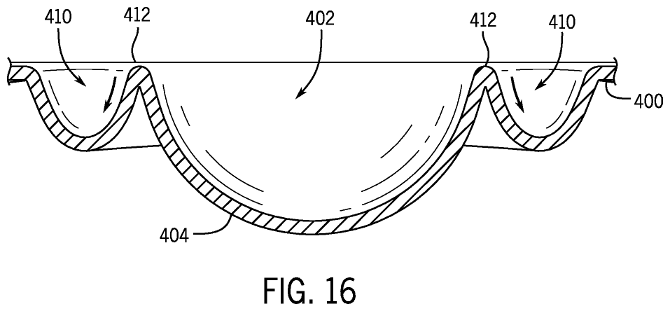

FIG. 9 illustrates a cross-sectional side view of the first mold plate 400 and second mold plate 500 of FIG. 8, with the second mold plate 500 in a position to mold the resin 200 in the first mold plate 400 according to one embodiment of the present disclosure. As shown in FIG. 9, the second mold plate 500 is shown pressed down on top of the first mold plate 400, such that the melted cannabis-derived resin 200 in the cavities 402 of the first mold plate 400 is pushed by the protrusions 502 and spreads out to the contours of the walls 404 of the cavities 402 of the first mold plate 400. A problem may occur if too much resin 200 is placed in the cavities 402 of the first mold plate 400 in that when the resin 200 is being pushed by the protrusions 502, the protrusions 502 may cause the resin 200 to spread not only to the contours of the walls 404 of the cavities 402, but also to spread out and across the surface of the first mold plate 400. This may result in the spreading resin 200 to combine and harden with similar spreading resin 200 of adjacent cavities 402. Once the excess resin 200 is cooled and hardened, this may result in outer shells being formed that are attached to one another by excess resin 200 and difficult to separate. Thus, as described below with reference to FIGS. 16-18, reservoirs may be formed in the first mold plate 400 to receive the excess resin 200 and prevent excess resin 200 from spreading out across the surface of the first mold plate 400.

The size and shape of the protrusions 502 in the second mold plate 500 are sized and shaped to determine and control the thickness of the shell to be formed. For example, the protrusions 502 can placed within the cavities 402 such that there is a predetermined, known distance between the walls of the protrusions 502 in the second mold plate 500 and the walls of the cavities 402 in the first mold plate 400, which will create a shell with a predetermined, known thickness. Thicknesses that may be used for the shell may depend on the desired application and known densities of cannabis-derived resins and inner materials, but can be, for example, from about 100 microns-1 mm thick. Since thickness of the shell corresponds to the overall weight of the dosing capsule, being able to design capsules with specified, predetermined wall thicknesses allows for more control of the overall weight of the finished dosing capsule.

The resin 200 is then allowed to cool within the cavities 402 and harden into a crystallized form, forming a shell 403 made of hardened or crystallized cannabis-derived resin 200 having the same general shape as the cavities 402 and the outer surface of the protrusions 502.

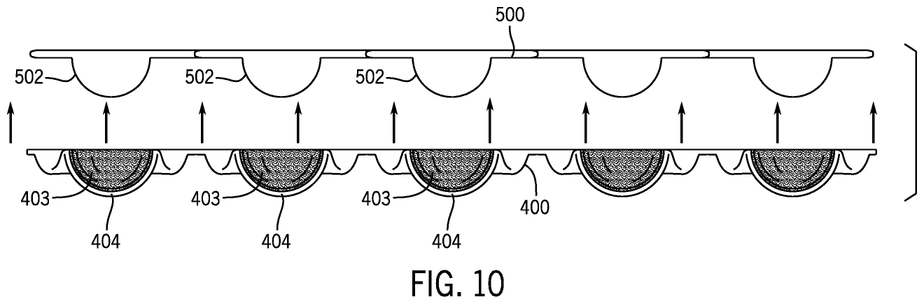

As shown in FIG. 10, the second mold plate 500 is then removed from the first mold plate 400, leaving the hardened shell 403 within the cavities 402 of the first mold plate 400.

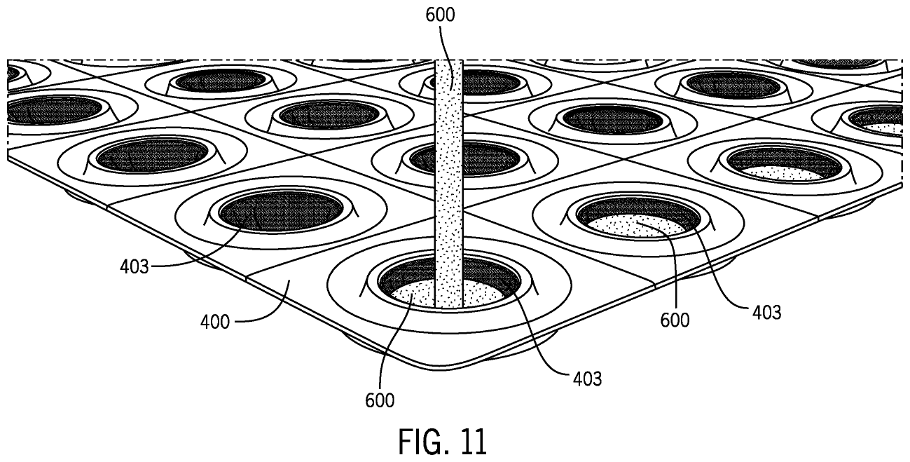

FIG. 11 shows an inner material 600 being dispensed into the hardened shells 403 in the cavities 402 of the first mold plate 400. The inner material 600 may be any ingestible material, such as a cannabis-derived resin, a pharmaceutical product or medication, or any other ingestible material that may be encapsulated such as a powder, oil or paste. The inner material 600 may be dispensed into the hardened cannabis-derived shells 403 using any dispensing method. A predetermined, known amount of the inner material 600 may be dispensed into each shell 403.

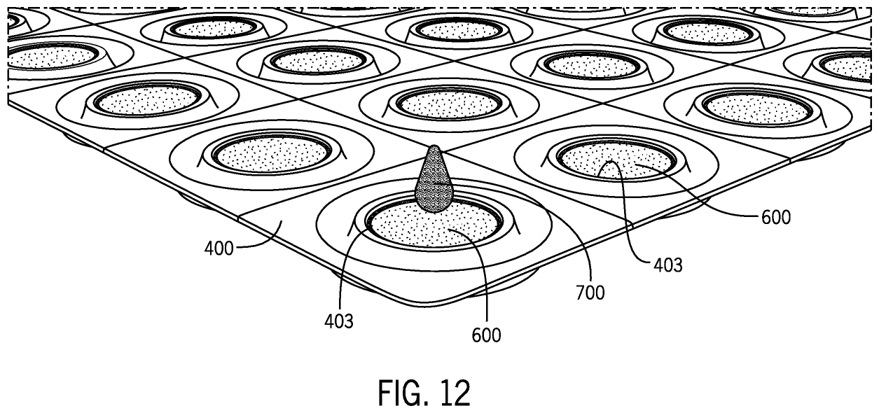

FIG. 12 shows a second resin 700 being added after the inner material 600 has been added to the shell 403. The second resin 700, which may also be a cannabis-derived resin and may also be the same resin as first resin 200, can be dispensed onto the hardened cannabis-derived resin shells 403 and over the inner material 600 within the shells 403. The second resin 700 may be heated into a melted state and dispensed using any dispensing method, now known or later developed. For instance, in one example, the second resin 700 may be manually dispensed onto each cavity by hand. In another example, an automated dispensing system may be used.

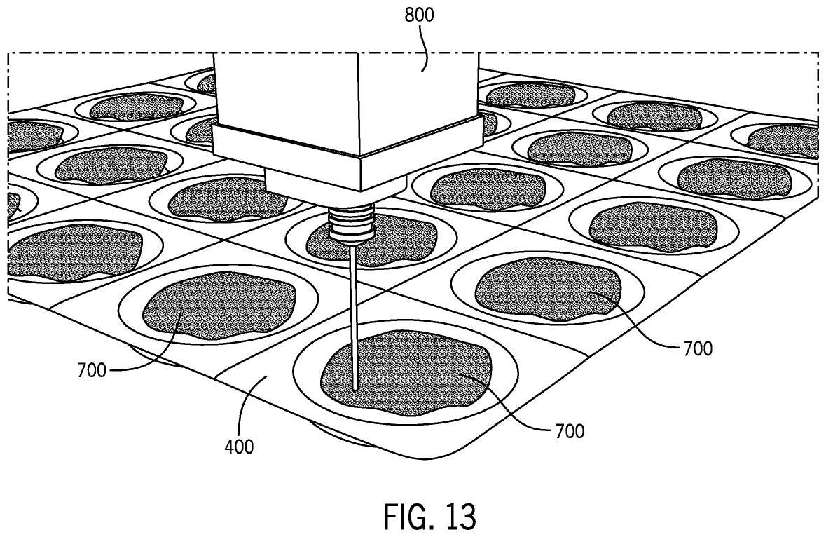

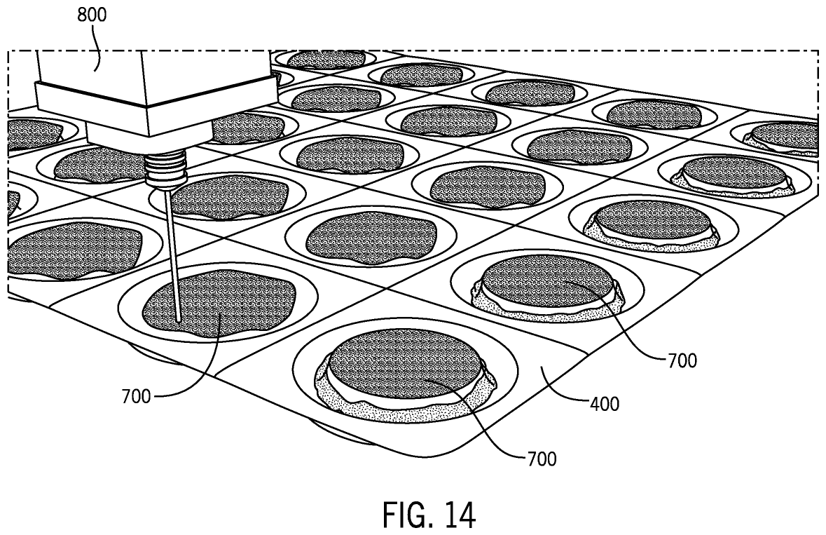

As shown in FIG. 12, an amount of the second resin 700 is dispensed onto the inner material 600 and is allowed to melt over the top of the shell 403 and inner material 600 (see FIGS. 13 and 14) and then cool. As the second resin 700 cools and hardens, it forms a seal over the shell 403 and inner material 600 and encapsulates the inner material 600 within the shell 403.

However, similar to the problem discussed above regarding excess resin 200, if too much second resin 700 is dispensed on top of the inner material 600 and hardened shell 403, the excess second resin 700 may spread towards other cavities 402 of the first mold plate 400 and combine with the shell 403, inner material 600, and/or excess second resin 700 being applied to that other cavity 402. If this happens and the excess second resin 700 is allowed to cool and harden, it can become very difficult to remove the induvial capsules created by the shells 403, inner material 600, and seal of second resin 700 in each cavity 402 of the first mold plate 400. Reservoirs surrounding the cavities 402 address this problem, as mentioned above and discussed below in greater detail with reference to FIGS. 16-18.

As shown in FIGS. 13 and 14, the second resin 700 may spread beyond the perimeter of the cavities 402 of the first mold plate 400 and over the perimeter of the shell 403 prior to cooling. After cooling, a laser 800 may be used to cut away the excess hardened second resin 700. In some examples, a die cutting tool, or any other cutting tool, may be used to cut away the excess hardened second resin 700. As shown in FIG. 13, the laser 800 is being used to cut away excess hardened second resin 700 on a first cavity 402 of the first mold plate 400. As shown in FIG. 14, the laser 800 has finished cutting away excess hardened second resin 700 of an entire row of cavities 402 of the first mold plate 400 and is beginning to start trimming excess hardened second resin 700 of a second row of cavities 402.

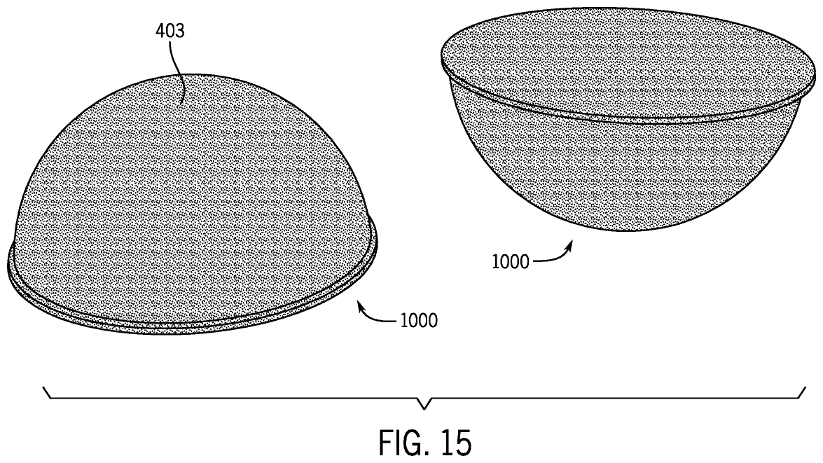

FIG. 15 illustrates perspective views of two dosing capsules made according to one embodiment of the present disclosure. As shown in FIG. 15, after the excess hardened second resin 700 is removed, the seal matches or closely matches the perimeter of the shell 403 and the sealed dosing capsules 1000 can be removed from the first mold plate 400. The dosing capsules 1000 shown in FIG. 15 may be ingested or consumed through vaporization, smoking, or used to make an edible product.

As mentioned above, if the top of the first mold plate 400 is designed to be a generally flat horizontal surface except for the cavities 402, a problem may occur with either excess resin 200 and/or excess second resin 700 combining and hardening with materials of adjacent cavities 402. In other words, referring back to FIGS. 8 and 9, once the cavities 402 are filled with resin 200 and the second mold plate 500 is placed on top of the first mold plate 400 so that the protrusions 502 are placed in the cavities 402 causing the resin 200 to spread out to the contours of the walls 404 of cavities 402 of the first mold plate 400, if there is too much resin 200 in the cavities 402 the resin 200 may spread out beyond the perimeter of the cavities 402 and combine and harden with resin 200 of adjacent cavities 402. As mentioned above with reference to FIG. 12, the same problem may occur with excess second resin 700. Having either of the resins 200, 700 spread out across the surface of the first mold plate 400 and combine with resins 200, 700 of adjacent cavities 402 makes it very difficult to remove the hardened shells 403 or finished sealed dosing capsules 1000 without damaging the shells 403 or capsules 1000.

One solution to the aforementioned problems is to provide a reservoir or trench-like area around the cavities 402 so that the excess resin 200 spreads downward into the reservoir/trench instead of horizontally across the surface of the first mold plate 400 as the resin 200 is pushed out of the cavities 402 by the protrusions 502. Since the resin 200 cools down and hardens very quickly, such as around 9-12 seconds after coming into contact with a cool surface such as a silicone mold like the first mold plate 400, the top mold plate (i.e., second mold plate 500) needs to be pressed down onto the bottom mold plate (i.e., first mold plate 400) quickly so that the resin 200 remains in liquid form. This way, the resin 200 is able to flow downward beyond the cavity 402 lip into the reservoir or trench. Thus, a thin lip of the cavities 402 and angled walls of the reservoir allow gravity to cause the resin 200 in liquid state to flow downward into the reservoir.

FIG. 16 shows one example of a cross-sectional side view of one of the cavities 402 of the first mold plate 400. As shown in FIG. 16, the cavity 402 may have a peripheral reservoir 410 that is used to catch excess resin 200 during the shell forming process and second resin 700 during the trimming process. Particularly, the peripheral reservoir 410 may be used to catch excess resin 200 that is pushed out of the cavity 402 by a corresponding protrusion 502. Similarly, the peripheral reservoir 410 may be used to catch excess second resin 700 that is trimmed by the laser 800 or other tool used to cut the excess hardened second resin 700. The reservoir 410 may be connected to the cavity 402 via a small lip or round 412 that forms a rim around the cavity 402. When the excess resin 200 is pushed out of the cavity 402 by a corresponding protrusion 502, the excess resin 200 flows over the lip or round 412 (i.e., rim) and down the walls of the reservoir 410 as shown by the arrows in FIG. 16. Similarly, when the excess hardened second resin 700 is cut (e.g., by a laser) it falls outwardly and downwardly into the peripheral reservoir 410 as it is removed from the dosing capsule 1000 (also as shown by the arrows in FIG. 16). The size and shape of the reservoirs 410 may vary. Although the reservoirs 410 of FIG. 16 are shown to be approximately half as deep as the cavity 402, the reservoirs 410 may be as deep or deeper than the cavity 402. Having deeper reservoirs 410 allows for a greater amount of excess material to be collected and prevents an abundance of excess material to flow either back into the cavity 402 or to nearby, adjacent cavities 402 to the extent both reservoirs 410 are full of excess material. Similarly, the width of the reservoirs 410 may be increased as well to accommodate additional excess material.

It is advantageous for the lip or round 412 to be very small so that there is very little surface area for the resin 200 to adhere to while flowing over the lip or round 412, leaving no place else to flow except downward into the reservoir 410. Another advantage of having a thin lip or round 412 is that to the extent there is resin 200 that happens to cool and harden on the top surface of the cavity rim (i.e., lip or round 412), the hardened resin 200 would be so thin at that location that it is easily breakable without damaging the rest of the dosing capsule 1000 or adjacent dosing capsules 1000, thus preventing the inner material 600 from spilling out. This also allows the adjacent dosing capsules 1000 to be easily separated from each other. In one example, the average thickness of the lip or round 412 may be between about 100-500 microns. In another example, the lip or round 412 may be between about 100-400 microns thick.

There are a number of advantages of collecting excess resin 200 and trimming excess hardened second resin 700 and collecting the excess resin 700 in the reservoir 410. For one, both allow for the end product to be a clean dosing capsule 1000 without excess material, which not only reduces the overall weight of the dosing capsule 1000, but also results in a more aesthetically pleasing dosing capsule 1000 having clean edges. Furthermore, capturing the excess resin and excess hardened second resin 700 allows for the excess resins 200, 700 to be reused, which results in less waste and a more overall efficient manufacturing process.

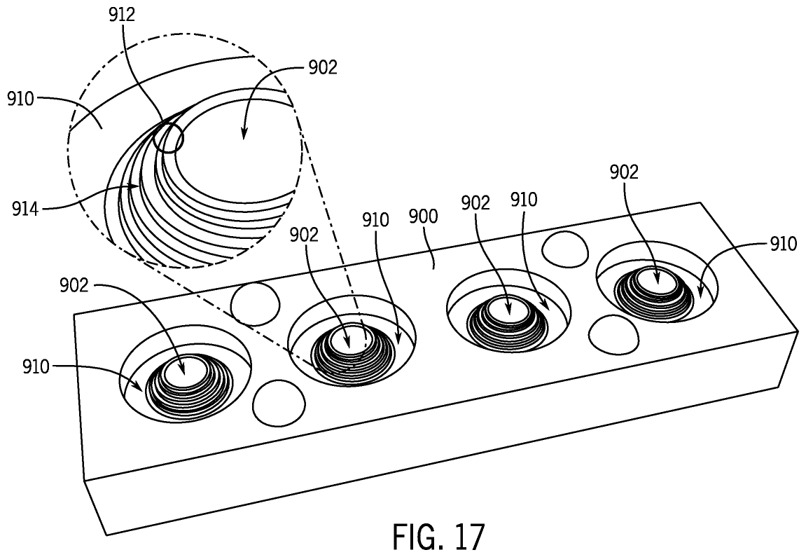

FIG. 17 shows a perspective view of another example of a first mold plate 900. As shown in FIG. 17, the first mold plate 900 has a number of cavities 902. While only four cavities 902 are shown in FIG. 17, any number of cavities 902 may be used. The cavities 902 are similar to the cavities 402 described above. The first mold plate 900 has peripheral reservoirs 910 around each cavity 902. The reservoirs 910 are used to catch excess resin 200 during the shell forming process and excess second resin 700 during either the dispensing of the second resin 700 or during the trimming process. Particularly, the reservoirs 910 may be used to catch excess resin 200 that is pushed out of the cavity 902 by a corresponding protrusion. Similarly, the reservoirs 910 may be used to catch the amount of excess second resin 700 that spreads and spills over the edge of the cavity 902 as it is dispensed or as it is trimmed by the laser 800 once hardened (or other tool used to cut the excess second resin 700). As shown in FIG. 17, each reservoir 910 is connected to a corresponding cavity 902 via a generally flat, generally horizontal wall 912, seen in more detail in the enlarged view of FIG. 17, such that when the excess resin 200 flows over the wall 912 or when the second resin 700 either flows over the wall 912 or is cut away or trimmed (e.g., by a laser), the excess resin 200, 700 flows or falls outwardly and downwardly into the peripheral reservoir 910 as it flows away from or is removed from the dosing capsule 1000.

In one example, the flat wall 912 may be about 10 to 2000 microns wide. For certain methods of trimming any excess second resin 700, such as where any excess second resin 700 is trimmed with a laser 800, the width of the flat wall 912 may be between about 500 microns and 1000 microns.

As shown in FIG. 17, there is also a generally vertical wall 914 made of steps within the inner portion of the reservoir 910. The flat wall 912 is located at the top step such that it acts as a rim of the cavity 902. The steps could be any shape or size that allows the excess resin or excess second resin 700 to flow or fall away from the flat wall 912 and down the vertical wall 914 into the reservoir 910, either by overflowing over the flat wall 912 and down the vertical wall 914 if too much resin 200 or second resin 700 is applied or by falling away from the flat wall 912 and down the vertical wall 914 during the trimming process. In another example, instead of steps the vertical wall 914 could be cylindrical, or any other shape that supports a flat wall 912 and allows the extra or cut excess resin 200, 700 to flow or fall into the reservoir 910.

FIG. 18 is a perspective view of another example of a first mold plate 950. In the example shown in FIG. 18, there are a number of cavities 952 in the first mold plate 950, each cavity 952 having a flat wall 964, similar to the cavities 902 and flat walls 912 shown in FIG. 17. In one example, the flat wall 964 may be about 10 to 2000 microns wide. For certain methods of trimming any excess second resin 700, such as where any excess second resin 700 is trimmed with a laser 800, the width of the flat wall 964 may be between about 500 microns and 1000 microns. However, there are a couple notable differences between the first mold plate 900 of FIG. 17 and the first mold plate 950 of FIG. 18. As shown in the example of FIG. 18, the vertical wall 954 is made of a conical wall portion (e.g., upper portion) and a cylindrical wall portion (e.g., lower portion), as opposed to the stepped vertical wall 914 of FIG. 17. Also, in the example shown in FIG. 18, a single reservoir 960 surrounds all of the cavities 952, as opposed to individual reservoirs 910 for each cavity 902 as in FIG. 17. In other examples, not shown, the reservoirs 910, 960 may surround some, but not all of the cavities 902, 952 of the first mold plates 900, 950. While only six cavities 952 are shown in FIG. 18, any number of cavities 952 may be used.

Similar to the reservoirs 910 of FIG. 17, the reservoir 960 of FIG. 18 is used to catch excess resin 200 during the shell forming process and excess second resin 700 during either the dispensing of the second resin 700 or during the trimming process. Particularly, the reservoir 960 may be used to catch excess resin 200 that is pushed out of the cavity 902 by a corresponding protrusion of another mold during the shell forming process. For example, once each cavity 952 of the first mold plate 950 is filled with resin 200, a second mold plate (not shown) having protrusions corresponding to the cavities 952 of the first mold plate 950 may be pressed down on top of the first mold plate 950. The second mold plate in this example may be designed to be sized similarly to the first mold plate 950 with protrusions along the center of the second mold plate that correspond to the cavities 952 of the first mold plate 950. The second mold plate may also have other cavities, indentations, or depressions at the corners of the second mold plate to correspond with the projections or protuberances 970 of the first mold plate 950 to ensure, and maintain, proper alignment of the two mold plates. In this way, when the second mold plate is placed on top of the first mold plate 950, the projections or protuberances 970 of the first mold plate 950 engage or nest within the corresponding cavities, indentations, or depressions at the corners of the second mold plate and the protrusions along the center of the second mold plate engage or nest within the corresponding cavities 952 of the first mold plate 950 containing the resin 200. Any excess resin 200 that spills out during this process flows out and over the wall 964, down the vertical wall 954, and into the reservoir 960 for collection and reuse.

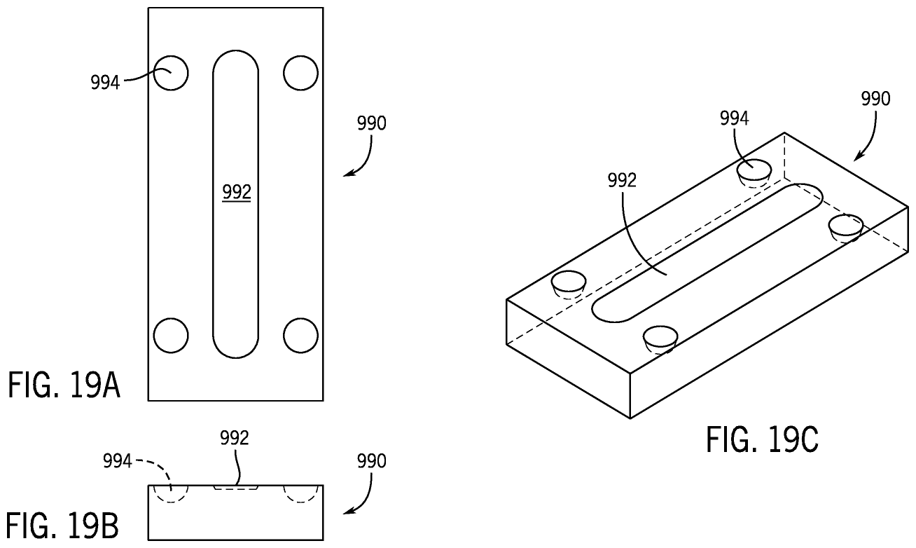

Similarly, the reservoir 960 may be used to catch any excess second resin 700 either during the dispensing of the second resin 700 or as it is trimmed by the laser 800 or other tool used to cut the excess second resin 700 during a trimming process after sealing the capsule 1000 as described above, or to catch any excess second resin 700 that flows out and over the wall 964 during an improved sealing process using a third mold, as described below with reference to FIGS. 19A-C. As discussed above with reference to FIG. 12, the sealing process involves a second resin 700 being added after the inner material 600 has been added to the shell 403. The second resin 700, which may also be a cannabis-derived resin and may also be the same resin as first resin 200, can be dispensed onto the hardened cannabis-derived resin shells 403 and over the inner material 600 within the shells 403. The second resin 700 may heated into a melted state and dispensed using any dispensing method. Generally, an amount of the second resin 700 is dispensed onto the inner material 600 and is allowed to melt over the top of the shell 403 and inner material 600 (see FIGS. 13 and 14) and then cool. As the second resin 700 cools and hardens, it forms a seal over the shell 403 and inner material 600 and encapsulates the inner material 600 within the shell 403.

However, while sealing using this method is effective in sealing the inner material 600 within the shell 403, this sealing method may result in variable overall weights of the dosing capsules 1000 since the second resin 700 may cool and harden at different heights or thicknesses after it is applied or dispensed. It may be possible to dispense the second resin 700 at specified, predetermined heights or to manually smooth out the second resin 700 to a uniform thickness after it is applied (e.g., using a tool and hand pressure applied to the resin 700), but it is difficult to manually ensure a uniform thickness. Additionally, since the second resin 700 is still in liquid form at this point, the second resin 700 may still be able to flow to uneven thicknesses if not constrained by some structure. The third mold described below provides this needed structure, which ensures a consistent, predetermined thickness of the seal.

FIGS. 19A, 19B, and 19C show a top view, cross-sectional side view, and perspective view, respectively, of a third mold plate 990 according to one embodiment of the present disclosure. As shown in FIGS. 19A-C, the third mold plate 990 is designed to correspond to a first mold plate, such as the first mold plate 950 of FIG. 18. The third mold plate 990 is sized to have similar dimensions of, and fit on top of, the first mold plate 950, similar to how the second mold plate described above fits on the first mold plate 950. The third mold plate 990 includes cavities, indentations, or depressions 994 near the corners of the third mold plate 990. The depressions 994 are sized to correspond to the protuberances 970 of the first mold plate 950, such that when the third mold plate 990 is placed on top of the first mold plate 950, the protuberances 970 of the first mold plate 950 engage or nest within the depressions 994 of the third mold plate 990 to ensure, and maintain, proper alignment of the two mold plates.

Also as shown in FIGS. 19A-C, the third mold plate 990 includes a central recess 992 spanning a length along the center of the third mold plate 990. In one example, the size and shape of the perimeter of the recess 992 may correspond to the size and shape of the perimeter of the reservoir 960 of the first mold plate 950 of FIG. 18. In this regard, when the third mold plate 990 is placed on top of the first mold plate 950 as described above, the recess 992 is positioned directly above the reservoir 960 of the first mold plate 950. The recess 992 may have a flat, depressed inner surface and a predetermined thickness or depth that corresponds to a desired thickness of the seal formed by the second resin 700, as discussed above. The depressed, inner surface of the recess 992 is flat to ensure a uniform outer surface of the resin seal described herein. In one example, the thickness or depth of the recess 992 may be chosen based on the thickness of the outer shell 403. For instance, if the outer shell 403 measures 400 microns thick, the thickness or depth of the recess 992 may be designed to be 400 microns as well to ensure the resin seal is also 400 microns. In this way, the inner material 600 may be surrounded by a uniform thickness of resin (e.g., 400 microns in this case). In another example, the thickness or depth of the recess 992, which corresponds to the thickness of the resin seal, does not need to be the same thickness as the outer shell 403. The thickness of both the outer shells 403 and resin seals may vary depending on a desired product and/or application. Use of the third mold plate 990 to ensure a consistent, predetermined thickness of the seal formed by the second resin 700 is described below with reference to FIGS. 18 and 19A-C.

Once the outer shells 403 are formed and filled with the inner material 600 as described above, the second resin 700 is dispensed or otherwise applied on top of the inner material 600 and outer shells 403 contained in the cavities 952 of the first mold plate 950 to create an encapsulation of the inner material 600. As soon as the second resin 700 is applied, the third mold plate 990 is pressed down on top of the first mold plate 950. As discussed above, the reason this needs to be performed quickly after dispensing the second resin 700 is because the second resin 700 may cool and harden quickly. As the third mold plate 990 is placed on top of the first mold plate 950, the second resin 700 on top of the inner material 600 and outer shells 403 comes into contact with the flat surface of the recess 992 of the third mold plate 990 and spreads outwardly and evenly across the top of the inner material 600 and outer shells 403 of each cavity 952 of the first mold plate 950, thereby forming a uniform thickness of second resin 700 over each filled cavity 952. As described above, any excess second resin 700 flows outwardly over the walls 964 and down the vertical walls 954 of each cavity 952 and into the reservoir 960 of the first mold plate 950 for collection and potential reuse. As the second resin 700 comes into contact with the surface of the recess 992 of the third mold plate 990, the second resin 700 cools and solidifies or hardens, thereby forming a uniform thickness resin seal encapsulating the inner material 600 inside the outer shells 403 of each cavity 952 of the first mold plate 950.

Once the resin seal is formed as described above, the third mold plate 990 is separated from the first mold plate 950 and the dosing capsules 1000 can be removed from the cavities 952 of the first mold plate 950. Any extra hardened resin along the perimeter of the dosing capsules 1000 that may have been caused by excess second resin 700 hardening to the thin walls 964 of the cavities 952 may be removed at this point. Since most of the excess second resin 700 would flow over the walls 964 and down the vertical walls 954 of each cavity 952, there should be very little, if any, second resin 700 left to harden directly on top of the walls 964 of the cavities 952. The amount of second resin 700 that does accumulate and harden on top of the walls 964 of the cavities 952 should be very thin, allowing it to be easily removed.

As discussed above, having a uniform thickness resin seal that is predetermined allows more control of the manufacturing process, and in particular more control over the amount of inner material 600 and overall weight and dimensions of the dosing capsule 1000. Additionally, the third mold plate 990 allows controlling the shape, size, and weight of the dosing capsule 1000 more precisely resulting in capsule dose and weight fluctuation within a much smaller range, and much more quickly, compared to known dosing capsules.

The third mold plate 990 may also be used to stamp or otherwise provide insignia, such as text, logos, shapes, holograms, and the like, onto the surface of the resin seal of the dosing capsules 1000. The desired insignia (or rather a negative of the desired insignia) may be formed into certain portions of the flat depressed, inner surface of the recess 992 that correspond to the cavities 952 of the first mold plate 950, such that when the third mold plate 990 is pressed down on top of the first mold plate 950, the inner surface of the recess 992 comes into contact with the second resin 700 on top of the inner material 600 and outer shells 403 and cools and hardens with the insignia formed therein.

In the case of holographic optical images, surface elements may be formed in the portions of the flat depressed, inner surface of the recess 992 that correspond to the cavities 952 of the first mold plate 950. The surface elements on the inner surface of the recess 992 form optical structures in the second resin 700 when the surface elements contact the second resin 700. The optical structures can be used to generate images with holographic effects.

While mold plates disclosed herein may be referred to as a first mold plate, second mold plate, and third mold plate, each of these mold plates may be individually or collectively referred to as a mold. In other words, a mold may include one or more mold plates or mold blocks.

Many different arrangements of the process of making and components described above, as well as components and steps not shown, are possible without departing from the spirit and scope of the present disclosure. The aforementioned method has been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

One aspect (“aspect one”) relates to a method of manufacturing dosing capsules from cannabis-derived resin using a mold system, the method comprising the steps of: providing a dispensing plate having one or more reservoirs for holding a cannabis-derived resin; providing a first mold plate having one or more cavities defined by walls corresponding to one or more reservoirs of the dispensing plate; providing a second mold plate having one or more protrusions corresponding to one or more cavities of the first mold plate; depositing into the reservoirs of the dispensing plate a first cannabis-derived resin in powder form; heating the first cannabis-derived resin such that the first cannabis-derived resin powder melts to become liquid; placing the first mold plate over the dispensing plate so that at least one reservoir of the dispensing plate registers with at least one cavity in the first mold plate; securing the first mold plate to the dispensing plate and rotating the dispensing plate and first mold plate, and transferring the liquid first cannabis-derived resin from the reservoirs of the dispensing plate into the cavities of the first mold plate; separating the dispensing plate from the first mold plate; placing a second mold plate, containing at least one protrusion that corresponds with at least one cavity of the first mold plate, on top of the first mold plate such that the liquid first cannabis-derived resin is pressed by the protrusion to a wall of the cavity of the first mold plate; allowing the first cannabis-derived resin to cool into a hardened shell; removing the second mold plate from the first mold plate; filling the hardened shell in the first mold plate with an inner material; and dispensing a second cannabis-derived resin over the hardened shell and inner material to seal the inner material within the hardened shell.

Another aspect (“aspect two”) relates to the method of aspect one, wherein the first cannabis-derived resin comprises tetrahydrocannabinol acid (THCA), cannabidiolic acid (CBDA), cannabigerolic acid (CBGA), or combinations thereof.

Another aspect (“aspect three”) relates to the method of aspect two, wherein the inner material comprises a cannabis derived resin, a pharmaceutical product, an essential oil, or combinations thereof.

Another aspect (“aspect four”) relates to the method of aspect two, wherein the second cannabis derived resin comprises THCA, CBDA, CBGA, or combinations thereof.

Another aspect (“aspect five”) relates to the method of aspect one, further comprising removing any amount of second cannabis-derived resin that has spilled outside of the cavities of the first mold plate.

Another aspect (“aspect six”) relates to the method of aspect one, further comprising placing a third mold plate, containing a recess having a predetermined thickness and a flat surface, on top of the first mold plate such that the second cannabis-derived resin is pressed by the flat surface of the recess to form a seal of uniform thickness.

Another aspect (“aspect seven”) relates to a dosing capsule having a shell made of a cannabis-derived resin formed by: providing a dispensing plate having one or more reservoirs for holding a cannabis-derived resin; providing a first mold plate having one or more cavities defined by walls corresponding to one or more reservoirs of the dispensing plate; providing a second mold plate having one or more protrusions corresponding to one or more cavities of the first mold plate; depositing into the reservoirs of the dispensing plate a first cannabis-derived resin in powder form; heating the first cannabis-derived resin such that the first cannabis-derived resin powder melts to become liquid; placing the first mold plate over the dispensing plate so that at least one reservoir of the dispensing plate registers with at least one cavity of the first mold plate; securing the first mold plate to the dispensing plate and rotating the dispensing plate and first mold plate, and transferring the liquid first cannabis-derived resin from the reservoirs of the dispensing plate into the cavities of the first mold plate; separating the dispensing plate from the first mold plate; placing a second mold plate, containing at least one protrusion that corresponds with at least one cavity of the first mold plate, on top of the first mold plate such that the liquid first cannabis-derived resin is pressed by the protrusion to a wall of the cavity of the first mold plate; allowing the first cannabis-derived resin to cool into a hardened shell; removing the second mold plate from the first mold plate; filling the hardened shell in the first mold plate with an inner material; and dispensing a second cannabis-derived resin over the hardened shell and inner material to seal the inner material within the hardened shell.

Another aspect (“aspect eight”) relates to the dosing capsule of aspect seven, further formed by placing a third mold plate, containing a recess having a predetermined thickness and a flat surface, on top of the first mold plate such that the second cannabis-derived resin is pressed by the flat surface of the recess to form a seal of uniform thickness.

Another aspect (“aspect nine”) relates to the dosing capsule of aspect seven, further formed by removing amounts of the second cannabis-derived resin that has spilled outside the cavities of the first mold plate.

Another aspect (“aspect ten”) relates to the dosing capsule of aspect seven, wherein the first cannabis-derived resin comprises THCA, CBDA, CBGA, or combinations thereof.

Another aspect (“aspect eleven”) relates to the dosing capsule of aspect seven, wherein the inner material comprises a cannabis derived resin, a pharmaceutical product, or combinations thereof.

Another aspect (“aspect twelve”) relates to the dosing capsule of aspect seven, wherein the second cannabis-derived resin comprises THCA, CBDA, CBGA, or combinations thereof.

Another aspect (“aspect thirteen”) relates to a mold for manufacturing a dosing capsule from a cannabis-derived resin comprising: a first mold block; a cavity formed within the first mold block; a reservoir surrounding the cavity; and a generally flat wall surrounding the cavity and separating the cavity from the reservoir.

Another aspect (“aspect fourteen”) relates to the mold for manufacturing a dosing capsule of aspect thirteen, wherein the width of the generally flat wall is between about 10 microns and 2000 microns (2 mm).

Another aspect (“aspect fifteen”) relates to the mold for manufacturing a dosing capsule of aspect thirteen, further comprising: a second mold block having a central recess of a predetermined depth, wherein the second mold block is configured to be placed on top of the first mold block such that the central recess is positioned above the cavity.

Another aspect (“aspect sixteen”) relates to the mold for manufacturing a dosing capsule of aspect thirteen, wherein the width of the generally flat wall is between about 500 microns and 1000 microns.

Another aspect (“aspect seventeen”) relates to a method of manufacturing dosing capsules from cannabis-derived resin using a mold system, the method comprising the steps of: providing a container for holding a cannabis-derived resin; providing a first mold plate having one or more cavities defined by walls; providing a second mold plate having one or more protrusions corresponding to one or more cavities of the first mold plate; depositing into the container a first cannabis-derived resin in powder form; heating the first cannabis-derived resin such that the first cannabis-derived resin powder melts to become liquid; transferring the liquid first cannabis-derived resin from the container into the cavities of the first mold plate; placing a second mold plate, containing at least one protrusion that corresponds with at least one cavity of the first mold plate, on top of the first mold plate such that the liquid first cannabis-derived resin is pressed by the at least one protrusion to a wall of the at least one cavity of the first mold plate; allowing the first cannabis-derived resin to cool into a hardened shell; removing the second mold plate from the first mold plate; filling the hardened shell in the first mold plate with an inner material; and dispensing a second cannabis-derived resin over the hardened shell and inner material to seal the inner material within the hardened shell.

Another aspect (“aspect eighteen”) relates to a method of manufacturing dosing capsules from cannabis-derived resin, the method comprising: dispensing a melted or liquid cannabis-derived resin into one or more cavities of a first mold plate; placing a second mold plate on top of the first mold plate, wherein the second mold plate contains at least one protrusion that corresponds with at least one cavity of the one or more cavities of the first mold plate, such that the melted or liquid cannabis-derived resin is forced to occupy a space between the at least one protrusion and the at least one cavity; allowing the melted or liquid cannabis-derived resin to cool into a hardened shell; removing the second mold plate from the first mold plate; filling the hardened shell in the first mold plate with an inner material; and dispensing another cannabis-derived resin over the hardened shell and inner material to seal the inner material within the hardened shell.

Another aspect (“aspect nineteen”) relates to the method of aspect seventeen, wherein the first cannabis-derived resin comprises tetrahydrocannabinol acid (THCA), cannabidiolic acid (CBDA), cannabigerolic acid (CBGA), or combinations thereof.

Another aspect (“aspect twenty”) relates to the method of aspect eighteen, wherein the melted or liquid cannabis-derived resin comprises tetrahydrocannabinol acid (THCA), cannabidiolic acid (CBDA), cannabigerolic acid (CBGA), or combinations thereof.

Another aspect (“aspect twenty-one”) relates to the method of aspect seventeen or aspect eighteen, wherein the inner material comprises a cannabis derived resin, a pharmaceutical product, an essential oil, or combinations thereof.

Another aspect (“aspect twenty-two”) relates to the method of aspect seventeen, wherein the second cannabis derived resin comprises THCA, CBDA, CBGA, or combinations thereof.

Another aspect (“aspect twenty-three”) relates to the method of aspect eighteen, wherein the other cannabis derived resin comprises THCA, CBDA, CBGA, or combinations thereof.

Another aspect (“aspect twenty-four”) relates to the method of aspect seventeen, further comprising placing a third mold plate on top of the first mold plate, wherein the third mold plate includes a recess having a predetermined thickness and a flat surface, such that the second cannabis-derived resin is pressed by the flat surface of the recess to form a seal of uniform thickness when the third mold plate is placed on top of the first mold plate.

Another aspect (“aspect twenty-five”) relates to the method of aspect eighteen, further comprising placing a third mold plate on top of the first mold plate, wherein the third mold plate includes a recess having a predetermined thickness and a flat surface, such that the other cannabis-derived resin is pressed by the flat surface of the recess to form a seal of uniform thickness when the third mold plate is placed on top of the first mold plate.

Another aspect (“aspect twenty-six”) relates to the method of aspect six, the dosing capsule of aspect eight, the method of aspect twenty-four, and the method of aspect twenty-five, wherein the flat surface of the recess includes insignia, such that when the cannabis-derived resin is pressed by the flat surface to form the seal, the insignia is formed into a surface of the seal.

What is claimed is:

1. A method of manufacturing dosing capsules from cannabis-derived resin using a mold system, the method comprising the steps of:
providing a dispensing plate having one or more reservoirs for holding a cannabis-derived resin;
providing a first mold plate having one or more cavities defined by walls corresponding to one or more reservoirs of the dispensing plate;
providing a second mold plate having one or more protrusions corresponding to one or more cavities of the first mold plate;
depositing into the reservoirs of the dispensing plate a first cannabis-derived resin in powder form;
heating the first cannabis-derived resin such that the first cannabis-derived resin powder melts to become liquid;
placing the first mold plate over the dispensing plate so that at least one reservoir of the dispensing plate registers with at least one cavity in the first mold plate;
securing the first mold plate to the dispensing plate and rotating the dispensing plate and first mold plate, and transferring the liquid first cannabis-derived resin from the reservoirs of the dispensing plate into the cavities of the first mold plate;
separating the dispensing plate from the first mold plate;
placing the second mold plate, containing at least one protrusion that corresponds with at least one cavity of the first mold plate, on top of the first mold plate such that the liquid first cannabis-derived resin is pressed by the protrusion to a wall of the cavity of the first mold plate;
allowing the first cannabis-derived resin to cool into a hardened shell;
removing the second mold plate from the first mold plate;
filling the hardened shell in the first mold plate with an inner material; and
dispensing a second cannabis-derived resin over the hardened shell and inner material to seal the inner material within the hardened shell.

2. The method of claim 1 wherein:
the first cannabis-derived resin comprises tetrahydrocannabinol acid (THCA), cannabidiolic acid (CBDA), cannabigerolic acid (CBGA), or combinations thereof.

3. The method of claim 2 wherein:
the inner material comprises a cannabis derived resin, a pharmaceutical product, an essential oil, or combinations thereof.

4. The method of claim 2 wherein:
the second cannabis derived resin comprises THCA, CBDA, CBGA, or combinations thereof.

5. The method of claim 1 further comprising:
removing any amount of second cannabis-derived resin that has spilled outside of the cavities of the first mold plate.

6. The method of claim 1 further comprising:
placing a third mold plate, containing a recess having a predetermined thickness and a flat surface, on top of the first mold plate such that the second cannabis-derived resin is pressed by the flat surface of the recess to form a seal of uniform thickness.

7. A dosing capsule having a shell made of a cannabis-derived resin formed by:
providing a dispensing plate having one or more reservoirs for holding a cannabis-derived resin;
providing a first mold plate having one or more cavities defined by walls corresponding to one or more reservoirs of the dispensing plate;
providing a second mold plate having one or more protrusions corresponding to one or more cavities of the first mold plate;
depositing into the reservoirs of the dispensing plate a first cannabis-derived resin in powder form;
heating the first cannabis-derived resin such that the first cannabis-derived resin powder melts to become liquid;
placing the first mold plate over the dispensing plate so that at least one reservoir of the dispensing plate registers with at least one cavity of the first mold plate;
securing the first mold plate to the dispensing plate and rotating the dispensing plate and first mold plate, and transferring the liquid first cannabis-derived resin from the reservoirs of the dispensing plate into the cavities of the first mold plate;
separating the dispensing plate from the first mold plate;
placing the second mold plate, containing at least one protrusion that corresponds with at least one cavity of the first mold plate, on top of the first mold plate such that the liquid first cannabis-derived resin is pressed by the protrusion to a wall of the cavity of the first mold plate;
allowing the first cannabis-derived resin to cool into a hardened shell;
removing the second mold plate from the first mold plate;
filling the hardened shell in the first mold plate with an inner material; and
dispensing a second cannabis-derived resin over the hardened shell and inner material to seal the inner material within the hardened shell.

8. The dosing capsule of claim 7 further formed by:
placing a third mold plate, containing a recess having a predetermined thickness and a flat surface, on top of the first mold plate such that the second cannabis-derived resin is pressed by the flat surface of the recess to form a seal of uniform thickness.

9. The dosing capsule of claim 7 further formed by:
removing amounts of the second cannabis-derived resin that has spilled outside the cavities of the first mold plate.

10. The dosing capsule of claim 7 wherein:
the first cannabis-derived resin comprises THCA, CBDA, CBGA, or combinations thereof.

11. The dosing capsule of claim 7 wherein:
the inner material comprises a cannabis derived resin, a pharmaceutical product, or combinations thereof.

12. The dosing capsule of claim 7 wherein:
the second cannabis-derived resin comprises THCA, CBDA, CBGA, or combinations thereof.

13. A mold for manufacturing a dosing capsule from a cannabis-derived resin comprising:
a first mold block;
a cavity formed within the first mold block;
a reservoir surrounding the cavity; and
a generally flat wall surrounding the cavity and separating the cavity from the reservoir.

14. The mold for manufacturing a dosing capsule from a cannabis-derived resin of claim 13, wherein the width of the generally flat wall is between about 10 microns and 2000 microns (2 mm).

15. The mold for manufacturing a dosing capsule from a cannabis-derived resin of claim 13, further comprising:
a second mold block having a central recess of a predetermined depth,
wherein the second mold block is configured to be placed on top of the first mold block such that the central recess is positioned above the cavity.
